(19) United States
(12) Patent Application Publication
(10) Pub. No.: US 2024/0420828 A1
(43) Pub. Date: Dec. 19, 2024

(54) METHOD FOR AUTOMATED WHOLE-SLIDE SCANNING OF GRAM STAINED SLIDES AND EARLY DETECTION OF MICROBIOLOGICAL INFECTION

(71) Applicant: SCOPIO LABS LTD., Tel Aviv (IL)

(72) Inventors: Ittai MADAR, Tel Aviv (IL); Ben LESHEM, Tel Aviv (IL); Erez NA'AMAN, Tel Aviv (IL); Eran SMALL, Yehud (IL)

(73) Assignee: SCOPIO LABS LTD., Tel Aviv (IL)

(21) Appl. No.: 18/705,277

(22) PCT Filed: Nov. 17, 2022
(86) PCT No.: PCT/IL2022/051225

§ 371 Date: Apr. 26, 2024

Related U.S. Application Data
(60) Provisional application No. 63/264,193, filed on Nov. 17, 2021.

(51) Int. Cl. G16H 30/40; G16H 10/40

(52) U.S. Cl. CPC G16H 30/40; G16H 10/40

(57) ABSTRACT

An apparatus configured to process a sample to detect a pathogen receives a slide with the sample on the slide, in which the sample has been stained with one or more of a Gram stain, an Acid-Fast stain, or a Giemsa stain. An area of at least 5 mm² of the sample is imaged at a rate of at least 15 mm² per minute and a resolution of 0.3 pm or better to generate one or more images. The one or more images of the sample are processed with a classifier configured to detect the pathogen in the sample. In some embodiments, a plurality of cultured and stained samples on a plurality of slides are imaged at the resolution and processed with the classifier, which can increase the area processed and analyzed in order to decrease the culture time and corresponding time to diagnose the patient.

### RELATED APPLICATIONS

This application claims the benefit under 35 U.S.C. § 119 (c) of U.S. Provisional Patent Application No. 63/264,193, filed Nov. 17, 2021, which is incorporated, in its entirety, by this reference.

### BACKGROUND

Prior approaches to diagnosing patients with pathogens such as blood pathogens can be less than ideal in at least some respects. For example, patients who have symptoms of infection may have very small amounts of the pathogen in their blood that may not be detectable with prior approaches. In some instances, a blood sample is cultured for days before the blood sample can be analyzed for pathogens, which can delay treatment such as treatment with an antibiotic. This delay can be harmful for the patient who may have worsening symptoms while treatment is delayed. Although the treating physicians may attempt to treat the patient with antibiotics, this can result in the patient receiving the incorrect treatment.

Although staining such as Gram, Acid-Fast, or a Giemsa staining can be used to analyze samples, at least some prior approaches generally rely on human expertise and culturing the sample for days prior to performing an analysis on the stained sample. Analysis of stained samples can be a complex, varied, time consuming process that is performed at the microbiology lab, and is one of the stages of the microbiological infection diagnosis. For example, the Gram stain differentiates groups of bacteria based on the color of the stain. Once a stained slide is prepared, it is placed under the microscope for manual examination. The observed color of the organism, along with its morphology and other cells, such as leukocytes, erythrocytes, epithelial cells, and others can be used in evaluating the sample. These manual processing and analysis techniques may require a high level of training and expertise and time for proper function. These factors, together with the high volumes of slides and samples that are evaluated leads to a consistent shortage of staff, long work hours, and fatigue, which may result in reduced sensitivity during stained sample tests.

Evaluation of some samples, such as microbiological organisms like bacteria, yeast, fungi, may rely on a high optical magnification and analysis of a relatively large sample area to reach a clinically valid conclusion. The time it takes to evaluate a slide in this manner, combined with a low concentration of an infectious organism in a sample, can result in failure to properly identify the existence of a microbiological organism, even when the sample has been cultured.

In light of the above, it would be beneficial to have improved methods, systems, apparatus and microscopes that ameliorate at least some of the aforementioned limitations of the prior approaches.

### SUMMARY

In some embodiments, the presently disclosed systems, methods and apparatus decrease the amount of time to detect a pathogen in a sample such as a cultured blood sample. In some embodiments, a sufficiently large area of a sample is imaged on a slide with a sufficiently high resolution and imaging rate to generate one or more images and the one or more images processed with a classifier to detect a pathogen, which can decrease the culture time of a sample such as a blood sample. In some embodiments, a plurality of cultured and stained samples on a plurality of slides are imaged at the resolution and processed with the classifier to detect the pathogen, which can increase the area processed and analyzed in order to decrease the culture time and corresponding time to diagnose the patient.

In some embodiments, the classification of the one or more images is performed simultaneously with the generation of the one or more images, which can decrease the time to generate a diagnosis. In some embodiments, a first processor is configured to generate the one or more images and a second processor is configured to processes the one or more images with the classifier, which can allow the processes to be performed simultaneously.

In some embodiments, the classification is performed with a plurality of separate processes, such as a plurality of threads, that allow the processing with the classifier to be performed more efficiently. In some embodiments, the separate classifier processes are allocated to different processor resources such as separate cores of a processor or arranged into sub-tasks that run the processes in parallel on the same processor or different processors. The classifier processes may comprise sub-tasks that can be arranged in a que of sub-tasks to be performed on the same processor or different processor, and combinations thereof. In some embodiments, a first classifier is configured to detect a first pathogen and a second classifier is configured to detect a second pathogen, and the classifiers run as parallel processes, such as separate processes on different processors or the same processor.

In some embodiments, a method of processing a sample to detect a pathogen comprises receiving a slide with the sample on the slide, wherein the sample has been stained with one or more of a Gram stain, an Acid-Fast stain, or a Giemsa stain. An area of at least 5 mm² of the sample is imaged at a rate of at least 15 mm² per minute and a resolution of 0.3 μm or better to generate one or more images. The one or more images of the sample are processed with a classifier configured to detect the pathogen in the sample.

### INCORPORATION BY REFERENCE

All patents, applications, and publications referred to and identified herein are hereby incorporated by reference in their entirety and shall be considered fully incorporated by reference even though referred to elsewhere in the application.

### BRIEF DESCRIPTION OF THE DRAWINGS

A better understanding of the features, advantages and principles of the present disclosure will be obtained by reference to the following detailed description that sets forth illustrative embodiments, and the accompanying drawings of which:

FIG. 1 shows a diagram of an exemplary microscope, in accordance with some embodiments;

FIG. 2 shows a method for automated whole-slide scanning of stained slides and early detection of microbiological infection, in accordance with some embodiments; and

FIG. 3 shows an exemplary computing system, in accordance with some embodiments; and

FIG. 4 shows an exemplary network architecture, in accordance with some embodiments.

### DETAILED DESCRIPTION

The following detailed description provides a better understanding of the features and advantages of the inventions described in the present disclosure in accordance with the embodiments disclosed herein. Although the detailed description includes many specific embodiments, these are provided by way of example only and should not be construed as limiting the scope of the inventions disclosed herein.

Disclosed herein is a digital, computer assisted system for quantitative whole-slide stain analysis from stains, which may be used as a decision support system and/or a digital platform for stain evaluation with remote consultation capabilities. The stain may comprise any suitable stain such as one or more of a Gram stain, an Acid-Fast stain, or a Giemsa stain, for example.

Some of the advantages of such a system include the use of digitalization and at least partial automation to reduce workloads, freeing technologists at the lab from the tedious process of manual microscopic evaluation of slides. Using digital scans and utilizing remote consultation to access decentralized expertise (e.g. in hospitals with less-skilled satellite sites), thereby allowing the experts to receive digital scans remotely and provide rapid, off-site analysis of Gram slides, while eliminating the need to physically ship slides. The use of a wide range of automatic analysis tools on digital imagery of the samples. In addition to detecting and classifying the infectious organisms within the slide, other tools may perform stain adequacy analysis related to stain quality, sample contamination analysis, and more.

The system may also provide a significant boost to early detection of low-concentration infections by detecting the presence of very few bacteria over whole slides. In the case of blood samples, the system may reduce the minimum culture time before knowing the sample is positive, or even making detection possible pre-culture, for example.

In the case of body fluids and other specimens, the sensitivity of pre-culture Gram tests may increase significantly, in part related to system's digital, whole-slide analysis.

The sample preparation techniques disclosed herein may be used to increase the bacteria concentration within the sample (e.g. microfluidic methods that filter out liquids and/or objects in the original sample that do not contain bacteria). This, in turn, can increase the effective concentration of bacteria in the outcome liquid (from which the slide is then prepared), thus increasing the effective number of bacteria present in the slide. Using these methods, decreased culture times may be achieved.

In addition, gathering vast amounts of digital data by scanning slides made from samples with known infections may allow training a system to automatically detect slight expressions, unique to certain organisms, in the scanned slides. This may yield better phenotyping options for both research and clinical purposes, allowing digital Gram analysis to classify bacteria with better accuracy.

The presently disclosed systems, methods and apparatuses are well suited for combination with prior approaches to analyzing samples such as blood samples. For example, the optical scanning apparatus may comprise one or more components of a conventional microscope with a sufficient numerical aperture, or a computational microscope as described in U.S. patent application Ser. No. 15/775,389, filed on Nov. 10, 2016, entitled “Computational microscopes and methods for generating an image under different illumination conditions,” published as US20190235224. The system may comprise one or more components of an autofocus system, for example as described in U.S. Pat. No. 10,705,326, entitled “Autofocus system for a computational microscope”. While the system may comprise any suitable user interface and data storage, in some embodiments, the system comprises one or more components for data storage and user interaction as described in U.S. Pat. No. 10,935,779, entitled “Digital microscope which operates as a server”. The system may comprise one or more components of an autoloader for loading slides, for example as described in U.S. patent application Ser. No. 16/875,665, filed on May 15, 2020, entitled “Multi/parallel scanner”. The system may comprise one or more components for selectively scanning areas of a sample, for example as described in U.S. patent application Ser. No. 16/875,721, filed on May 15, 2020, entitled “Accelerating digital microscopy scans using empty/dirty area detection,” published as US20200278530. The system may comprise a grid with a known pattern to facilitate image reconstruction, for example as described in U.S. Pat. No. 10,558,029, entitled “System for image reconstruction using a known pattern”. Each of the aforementioned patents and applications is incorporated herein by reference.

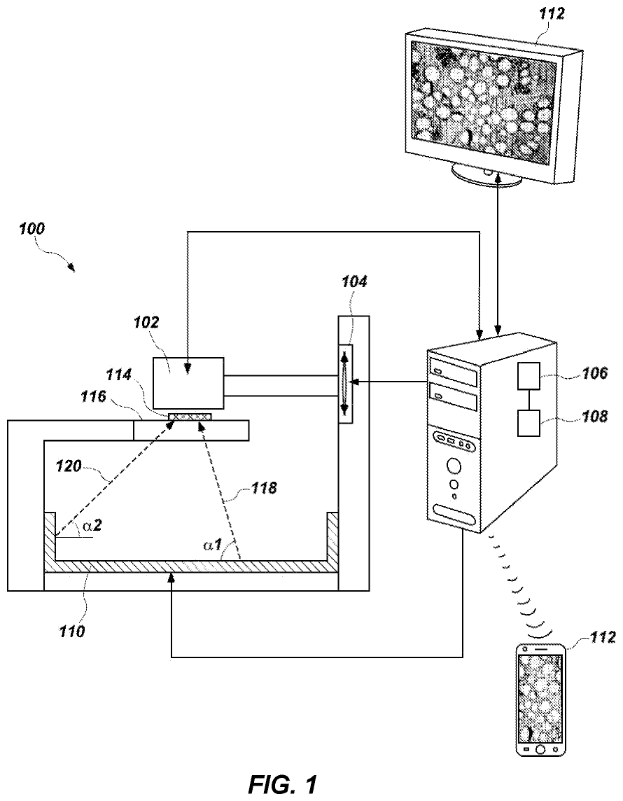

FIG. 1 is a diagrammatic representation of a microscope 100 consistent with the exemplary disclosed embodiments. The term “microscope” as used herein generally refers to any device or instrument for magnifying an object which is smaller than easily observable by the naked eye, i.e., creating an image of an object for a user where the image is larger than the object. One type of microscope may be an “optical microscope” that uses light in combination with an optical system for magnifying an object. An optical microscope may be a simple microscope having one or more magnifying lens. Another type of microscope may be a “computational microscope” that comprises an image sensor and image-processing algorithms to enhance or magnify the object's size or other properties. The computational microscope may be a dedicated device or created by incorporating software and/or hardware with an existing optical microscope to produce high-resolution digital images. As shown in FIG. 1, microscope 100 comprises an image capture device 102, a focus actuator 104, a controller 106 connected to memory 108, an illumination assembly 110, and a user interface 112. An example usage of microscope 100 may be capturing images of a sample 114 mounted on a stage 116 located within the field-of-view (FOV) of image capture device 102, processing the captured images, and presenting on user interface 112 a magnified image of sample 114.

Image capture device 102 may be used to capture images of sample 114. In this specification, the term “image capture device” as used herein generally refers to a device that records the optical signals entering a lens as an image or a sequence of images. The optical signals may be in the near-infrared, infrared, visible, and ultraviolet spectrums. Examples of an image capture device comprise a CCD camera, a CMOS camera, a color camera, a photo sensor array, a video camera, a mobile phone equipped with a camera, a webcam, a preview camera, a microscope objective and detector, etc. Some embodiments may comprise only a single image capture device 102, while other embodiments may comprise two, three, or even four or more image capture devices 102. In some embodiments, image capture device 102 may be configured to capture images in a defined field-of-view (FOV). Also, when microscope 100 comprises several image capture devices 102, image capture devices 102 may have overlap areas in their respective FOVs. Image capture device 102 may have one or more image sensors (not shown in FIG. 1) for capturing image data of sample 114. In other embodiments, image capture device 102 may be configured to capture images at an image resolution higher than VGA, higher than 1 Megapixel, higher than 2 Megapixels, higher than 5 Megapixels, 10 Megapixels, higher than 12 Megapixels, higher than 15 Megapixels, or higher than 20 Megapixels. In addition, image capture device 102 may also be configured to have a pixel size smaller than 15 micrometers, smaller than 10 micrometers, smaller than 5 micrometers, smaller than 3 micrometers, or smaller than 1.6 micrometer.

In some embodiments, microscope 100 comprises focus actuator 104. The term “focus actuator” as used herein generally refers to any device capable of converting input signals into physical motion for adjusting the relative distance between sample 114 and image capture device 102. Various focus actuators may be used, including, for example, linear motors, electrostrictive actuators, electrostatic motors, capacitive motors, voice coil actuators, magnetostrictive actuators, etc. In some embodiments, focus actuator 104 may comprise an analog position feedback sensor and/or a digital position feedback element. Focus actuator 104 is configured to receive instructions from controller 106 in order to make light beams converge to form a clear and sharply defined image of sample 114. In the example illustrated in FIG. 1, focus actuator 104 may be configured to adjust the distance by moving image capture device 102.

However, in other embodiments, focus actuator 104 may be configured to adjust the distance by moving stage 116, or by moving both image capture device 102 and stage 116. Microscope 100 may also comprise controller 106 for controlling the operation of microscope 100 according to the disclosed embodiments. Controller 106 may comprise various types of devices for performing logic operations on one or more inputs of image data and other data according to stored or accessible software instructions providing desired functionality. For example, controller 106 may comprise a central processing unit (CPU), support circuits, digital signal processors, integrated circuits, cache memory, or any other types of devices for image processing and analysis such as graphic processing units (GPUs). The CPU may comprise any number of microcontrollers or microprocessors configured to process the imagery from the image sensors. For example, the CPU may comprise any type of single- or multi-core processor, mobile device microcontroller, etc. Various processors may be used, including, for example, processors available from manufacturers such as Intel®, AMD®, etc. and may comprise various architectures (e.g., x86 processor, ARM®, etc.). The support circuits may be any number of circuits generally well known in the art, including cache, power supply, clock and input-output circuits. Controller 106 may be at a remote location, such as a computing device communicatively coupled to microscope 100.

In some embodiments, controller 106 may be associated with memory 108 used for storing software that, when executed by controller 106, controls the operation of microscope 100. In addition, memory 108 may also store electronic data associated with operation of microscope 100 such as, for example, captured or generated images of sample 114. In one instance, memory 108 may be integrated into the controller 106. In another instance, memory 108 may be separated from the controller 106.

Specifically, memory 108 may refer to multiple structures or computer-readable storage mediums located at controller 106 or at a remote location, such as a cloud server. Memory 108 may comprise any number of random-access memories, read only memories, flash memories, disk drives, optical storage, tape storage, removable storage and other types of storage.

Microscope 100 may comprise illumination assembly 110. The term “illumination assembly” as used herein generally refers to any device or system capable of projecting light to illuminate sample 114.

Illumination assembly 110 may comprise any number of light sources, such as light emitting diodes (LEDs), LED array, lasers, and lamps configured to emit light, such as a halogen lamp, an incandescent lamp, or a sodium lamp. For example, illumination assembly 110 may comprise a Kohler illumination source. Illumination assembly 110 may be configured to emit polychromatic light. For instance, the polychromatic light may comprise white light.

In some embodiments, illumination assembly 110 may comprise only a single light source. Alternatively, illumination assembly 110 may comprise four, sixteen, or even more than a hundred light sources organized in an array or a matrix. In some embodiments, illumination assembly 110 may use one or more light sources located at a surface parallel to illuminate sample 114. In other embodiments, illumination assembly 110 may use one or more light sources located at a surface perpendicular or at an angle to sample 114.

In addition, illumination assembly 110 may be configured to illuminate sample 114 in a series of different illumination conditions. In one example, illumination assembly 110 may comprise a plurality of light sources arranged in different illumination angles, such as a two-dimensional arrangement of light sources. In this case, the different illumination conditions may comprise different illumination angles. For example, FIG. 1 depicts a beam 118 projected from a first illumination angle α1, and a beam 120 projected from a second illumination angle α2. In some embodiments, first illumination angle α1 and second illumination angle α2 may have the same value but opposite sign. In other embodiments, first illumination angle α1 may be separated from second illumination angle α2. However, both angles originate from points within the acceptance angle of the optics. In another example, illumination assembly 110 may comprise a plurality of light sources configured to emit light in different wavelengths. In this case, the different illumination conditions may comprise different wavelengths. For instance, each light source may be configured to emit light with a full width half maximum bandwidth of no more than 50 nm so as to emit substantially monochromatic light. In yet another example, illumination assembly 110 may be configured to use a number of light sources at predetermined times. In this case, the different illumination conditions may comprise different illumination patterns. For example, the light sources may be arranged to sequentially illuminate the sample at different angles to provide one or more of digital refocusing, aberration correction, or resolution enhancement. Accordingly and consistent with the present disclosure, the different illumination conditions may be selected from a group including different durations, different intensities, different positions, different illumination angles, different illumination patterns, different wavelengths, or any combination thereof.

Although reference is made to computational microscopy, the presently disclosed systems and methods are well suited for use with many types of microscopy and microscopes such as one or more of a high definition microscope, a digital microscope, a scanning digital microscope, a 3D microscope, a phase imaging microscope, a phase contrast microscope, a dark field microscope, a differential interference contrast microscope, a light-sheet microscope, a confocal microscope, a holographic microscope, or a fluorescence-based microscope.

In some embodiments, image capture device 102 may have an effective numerical aperture (“NA”) of at least 0.8, although any effective NA may be used. In some embodiments, the effective NA corresponds to a resolving power of the microscope that has the same resolving power as an objective lens with that NA under relevant illumination conditions. Image capture device 102 may also have an objective lens with a suitable NA to provide the effective NA, although the NA of the objective lens may be less than the effective NA of the microscope. For example, the imaging apparatus may comprise a computational microscope to reconstruct an image from a plurality of images captured with different illumination angles as described herein, in which the reconstructed image corresponds to an effective NA that is higher than the NA of the objective lens of the image capture device. In some embodiments with conventional microscopes, the NA of the microscope objective corresponds to the effective NA of the images. The lens may comprise any suitable lens such as an oil immersion lens or a non-oil immersion lens.

Consistent with disclosed embodiments, microscope 100 may comprise, be connected with, or in communication with (e.g., over a network or wirelessly, e.g., via Bluetooth) user interface 112. The term “user interface” as used herein generally refers to any device suitable for presenting a magnified image of sample 114 or any device suitable for receiving inputs from one or more users of microscope 100. FIG. 1 illustrates two examples of user interface 112. The first example is a smartphone or a tablet wirelessly communicating with controller 106 over a Bluetooth, cellular connection or a Wi-Fi connection, directly or through a remote server. The second example is a PC display physically connected to controller 106. In some embodiments, user interface 112 may comprise user output devices, including, for example, a display, tactile device, speaker, etc. In other embodiments, user interface 112 may comprise user input devices, including, for example, a touchscreen, microphone, keyboard, pointer devices, cameras, knobs, buttons, etc. With such input devices, a user may be able to provide information inputs or commands to microscope 100 by typing instructions or information, providing voice commands, selecting menu options on a screen using buttons, pointers, or eye-tracking capabilities, or through any other suitable techniques for communicating information to microscope 100. User interface 112 may be connected (physically or wirelessly) with one or more processing devices, such as controller 106, to provide and receive information to or from a user and process that information. In some embodiments, such processing devices may execute instructions for responding to keyboard entries or menu selections, recognizing and interpreting touches and/or gestures made on a touchscreen, recognizing and tracking eye movements, receiving and interpreting voice commands, etc.

Microscope 100 may also comprise or be connected to stage 116. Stage 116 comprises any horizontal rigid surface where sample 114 may be mounted for examination. Stage 116 may comprise a mechanical connector for retaining a slide containing sample 114 in a fixed position. The mechanical connector may use one or more of the following: a mount, an attaching member, a holding arm, a clamp, a clip, an adjustable frame, a locking mechanism, a spring or any combination thereof. In some embodiments, stage 116 may comprise a translucent portion or an opening for allowing light to illuminate sample 114. For example, light transmitted from illumination assembly 110 may pass through sample 114 and towards image capture device 102. In some embodiments, stage 116 and/or sample 114 may be moved using motors or manual controls in the XY plane to enable imaging of multiple areas of the sample.

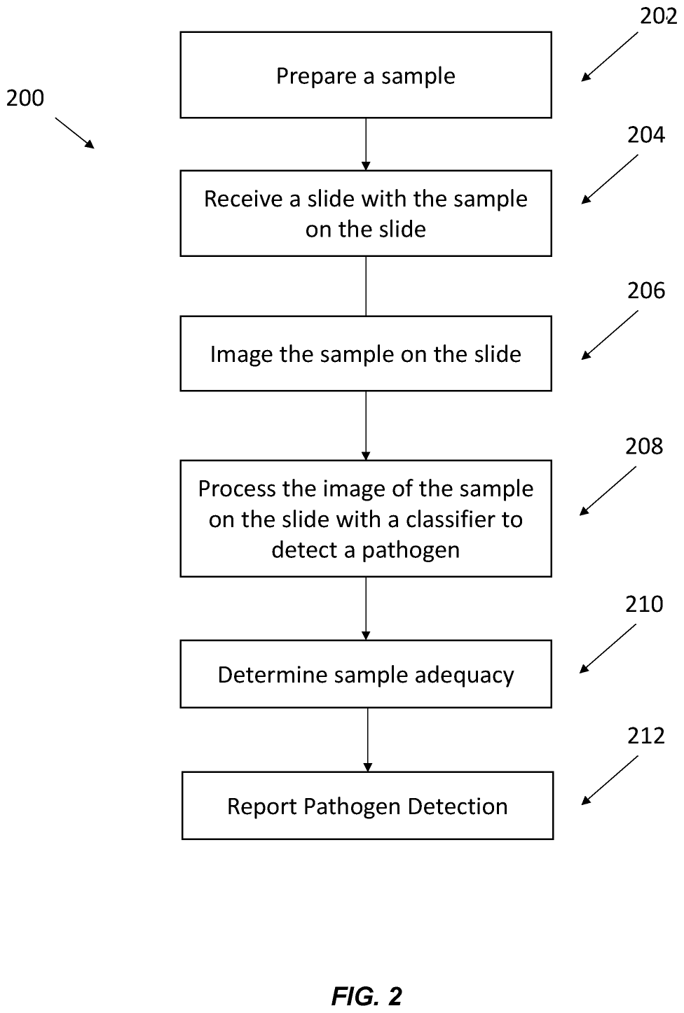

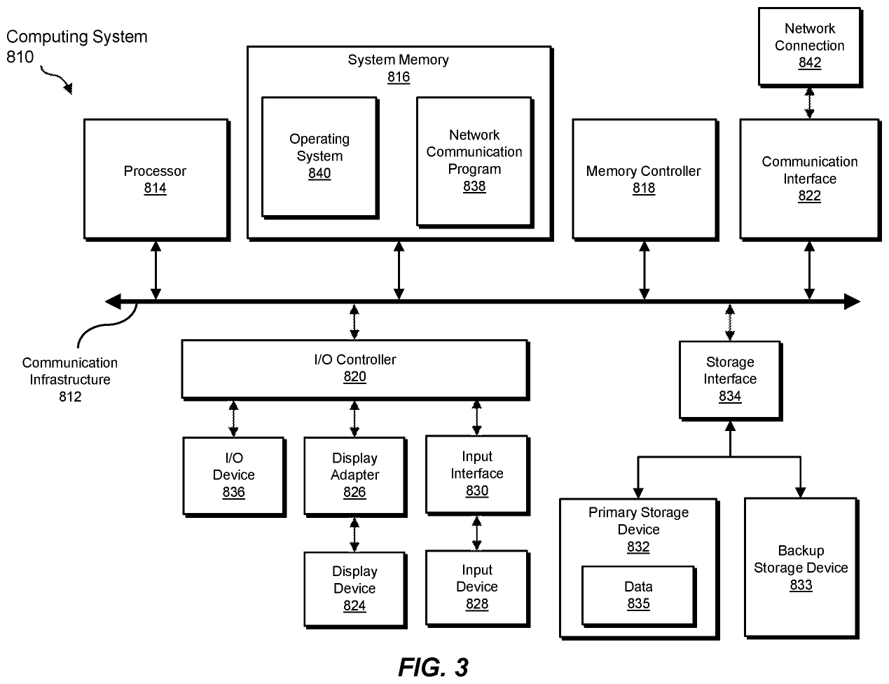

FIG. 2 is a flow diagram of an exemplary computer-implemented method 200 for automated whole-slide scanning of Gram stained slides and early detection of microbiological infection. The steps shown in FIG. 2 may be performed by a microscope system, such as the system(s) illustrated in FIGS. 2, 3, and/or 4. In one example, each of the steps shown in FIG. 2 may represent an algorithm whose structure includes and/or is represented by multiple sub-steps, examples of which will be provided in greater detail below.

A person of ordinary skill in the art will recognize that method 200 can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed. Also, some of the steps may be performed sequentially or at least partially simultaneously, for example. Further, some of the steps may be omitted, some of the steps repeated, and some of the steps may comprise substeps of other steps. Any of the steps may be combined with an input from a user, such as a remote user or a user operating the system, for example.

As illustrated in FIG. 2, at step 202 a sample is prepared. FIG. 3 provides additional details on how a sample may be received and prepared for imaging and classification. In some embodiments, the sample may be a human blood sample. In some embodiments, the sample may be an animal blood sample. In some embodiments, the sample may be drawn from a human or animal patient. In some embodiments, the sample may be received from someone who has drawn the sample from the patient. The sample can be collected in many ways, for example with a vial of blood. In some embodiments, two vials of blood are collected, a first vial for culturing a first blood sample for a first amount of time and a second vial collected for culturing a second sample for a second amount of time. This approach can allow for different testing approaches to be used, in which the first sample from the first vial is evaluated earlier than the second sample from the second vial, in order to potentially decrease the time to make a diagnosis. This approach also allows for combination of the rapid testing as described herein with traditional tests. In some embodiments, the sample may be collected from one or more of a blood, a plasma, a bodily fluid, a cerebrospinal fluid, a synovial fluid, a pleural fluid, a sputum, a mucus, an excrement, urine, an aspirate, a biopsy, or a swab or other sample type.

In some embodiments, the sample may comprise one or more of a bacterium, a fungus or a yeast. A blood sample may be cultured. Culturing a blood sample is a process by which blood is mixed with culturing agents and placed into an environment to promote the growth of pathogens, such as bacterium, fungus, or yeast. In some embodiments, the culture may be an aerobic blood culture. In some embodiments, the culture may be an anerobic blood culture. The atmosphere in a blood culture may include varying amounts of carbon dioxide, oxygen, and pH level. The culture medium may comprise a nutrient broth, for example. In some embodiments, the medium may comprise a general cultivation media, a selective media, a differential media, or a transport media. In some embodiments, the blood is cultured in a medium to grow a pathogen. The blood sample may be cultured for a period of time. In some embodiments, the blood is cultured in a culture medium for less than 48 hours. In some embodiments, the blood is cultured in a medium for no more than 24 hours. In some embodiments, the blood is cultured in a medium for no more than 1 hour, 2 hours, 4 hours, 8 hours, or 16 hours, for example.

In some embodiments, a plurality of vials or multiple sets of blood vials may be drawn from a single patient. In some embodiments, a first set of vials or a first vial of blood may be taken from a patient. A blood sample from the first vial or sets of vials may be cultured in a culture medium. In some embodiments, a second set of vials or a second vial of blood may be taken from the patient. A second blood sample from the second vial may be cultured in a second culture medium. In some embodiments, the first blood sample and the second blood sample may be cultured at the same or overlapping times, e.g. simultaneously. In some embodiments, a first blood sample may undergo scanning of Gram stained slides and early detection of microbiological infection, such as in steps 204, 206, and 208, while the second culture medium continues to grow the pathogen in the second sample. In some embodiments, the first sample may undergo automated whole-slide scanning of Gram stained slides and early detection of microbiological infection, such as in steps 204, 206, and 208, before the sample has incubated in the second culture medium for 40 hours. In some embodiments, the first sample is cultured for no more than 1 hour, 2 hours, 4 hours, 8 hours, or 16 hours before undergoing automated whole-slide scanning of Gram stained slides and early detection of microbiological infection, such as in steps 204, 206, and 208, while the second sample is cultured for less than 40 hours before undergoing automated whole-slide scanning of Gram stained slides and early detection of microbiological infection, such as in steps 204, 206, and 208.

In some embodiments, preparing the sample may include exposing a cultured ample to an antibiotic to perform an antibiotic sensitivity test.

After culturing the sample, the cultured sample, or a portion of the cultured sample may be placed on or more slides for imaging, as described herein.

In some embodiments, at block 202, the sample is stained with one or more of a Gram stain, an Acid-Fast stain, or a Giemsa stain. A Gram stain may be negative, in which case the stain is red or pink in color, or positive, in which case the stain is purple in color. In some embodiments, the received sample comprises a cultured sample in which a blood sample has been cultured in culture medium to grow the pathogen, for example as described with respect to block 202. In some embodiments, the blood sample has been cultured in a culture medium for less than 48 hours to grow the pathogen in the cultured sample, for example as described with respect to block 202. In some embodiments, the blood sample has been cultured in a culture medium for no more than 24 hours. In some embodiments, the blood sample has been cultured in a culture medium for no more than 3 hours, 6 hours, 12 hours, or 18 hours. In some embodiments, the blood sample has been cultured in the culture medium for no more than one or more of 1 hour, 2 hours, 4 hours, 8 hours, or 16 hours, for example.

At block 204 a slide is received with the prepared sample on the slide. In some embodiments, the sample has been stained with one or more of a Gram stain, an Acid-Fast stain, or a Giemsa stain. The sample may be stained with one or more of the stains in order to detect the presence or absence of one or more different pathogens. In some embodiments, a plurality of samples on a plurality of slides are received. The plurality of samples may be from a single patient, or a plurality of patients, and combinations thereof. Pathogens may only exist in very small quantities in samples, even cultured samples. A single vial of blood may be on the order of 10 mL to 15 mL while the volume of blood on a slide may be 10 μL to 15 μL. In some embodiments, a plurality of slides, such as at least 10 slides, at least 100 slides or at least 50 slides may be received with samples from a single patient. This approach can proportionally increase the area of the samples that are analyzed from the single patient at the resolution and rate as described herein. For example, if the area of the sample from a single slide that is imaged comprises one or more of at least 5 mm², at least 7.5 mm², at least 10 mm², at least 20 mm², at least 30 mm², at least 50 mm², or at least 70 mm², and 10 slides from a single patient are imaged and processed, the corresponding area comprises one or more of at least 50 mm², at least 75 mm², at least 100 mm², at least 200 mm², at least 300 mm², at least 500 mm², or at least 700 mm², for example. This approach can significantly increase the sensitivity and the specificity of the pathogen detection and can decrease the culture time for the sample that is placed on the slides for example.

In some embodiments, the plurality of samples may be taken from a plurality of different patients in order to batch process the imaging and classification processing of samples of a plurality of patients at the same time. In some embodiments, a subset of the plurality of samples has been taken form a single patient. For example, the samples from the same patient may have different cultured differently and/or stained differently. In some embodiments, the subset includes at least two samples from a single patient, for example.

In some embodiments, at least 30 samples from a single patient or a plurality of patients are received. In some embodiments, at least 30 samples are images and processed with the classifier, such as described with reference to block 206 and block 208 within one hour.

In some embodiments, the received sample may be a cultured sample that has been exposed to an antibiotic to perform an antibiotic sensitivity test, which can be compared to a culture sample that has not been exposed to the antibiotic. In some embodiments, the antibiotic sensitivity test may be carried out by imaging and processing the images in a classifier as discussed with respect to blocks 206 and 208 to detect the effectiveness of an antibiotic by detecting and comparing the number or amount of detected pathogens to a cultured sample that did not have the antibiotic, for example.

At block 206 the sample is imaged. In some embodiments, an area of at least 5 mm² of the sample is imaged at a rate of at least 15 mm² per minute at a resolution of 0.3 μm or better to generate one or more images. The one or more images may have an appropriate effective numerical aperture, such as at least one or more of 0.8, 0.9 or 1, for example. In some embodiments, the effective numerical aperture may be achieved by using a high numerical aperture lens in air. In some embodiments, a high numerical aperture may be achieved by using index matching material such as immersion oil or water. In some embodiments, a high numerical aperture may be achieved by using a lower numerical aperture index lens with computational methods that lead to a higher effective numerical aperture as described herein. In some embodiments, a high numerical aperture may be achieved by using a lens-less computational architecture, for example.

Imaging the sample may include imaging a scan area of the sample that is large enough to allow for detecting the existence of even a few infectious organisms anywhere on the slide. In some embodiments, the scanner is configured to scan the standard area of at least 50 high-power fields, such as 100× magnification fields, which are roughly equivalent to an area of at least 2 mm². In some embodiments the scan area may be larger, such as one or more of at least 3 mm², at least 5 mm², at least 10 mm², at least 50 mm², at least 100 mm², at least 2 cm², at least 2 cm², at least 10 cm², or at least 15 cm², for example.

In some embodiments, the imaging is carried out in a relatively short period of time. For example, in some embodiments, the imaging of the scan area occurs at a rate of at least 1 minute per cm².

In some embodiments, the sample may be imaged at a rate of at least 15 mm² per minute for at least a portion of the sample. In some embodiments, the sample may be imaged a rate of at least 18 mm² per minute, at least 20 mm² per minute, at least 25 mm² per minute, 50 mm² per minute, or 75 mm² per minute. In some embodiments, at least a portion of the ample may be images at a rate of at least 15 mm² per minute, at least 18 mm² per minute, at least 20 mm² per minute, at least 25 mm² per minute, 50 mm² per minute, or 75 mm² per minute.

In some embodiments, an area at least 7.5 mm², at least 10 mm², at least 20 mm², at least 30 mm², at least 50 mm², or at least 70 mm² of the sample is imaged at one or more of the rates described herein. In some embodiments, the sample area may be divided up among multiple areas on a single slide or among a plurality of slides.

In some embodiments, the sample is imaged with a resolution of at least 0.22 μm or better. In some embodiments, the sample is imaged with a resolution of at least 0.25 μm or better. In some embodiments, the resolution corresponds to the minimum distance at which an image of lines on a resolution target can be separated. In some embodiments, better image resolution corresponds to a smaller number for the smallest resolvable distance of features in an image.

In some embodiments, the one or more images generated at block 204 may be generated with one or more imaging techniques. In some embodiments, the imaging techniques may include computational photography, computational imaging, computational microscopy, ptychography or Fourier ptychography, as discussed herein, for example, with respect to FIG. 1. In some embodiments, the image may be generated from a plurality of imaging conditions. For example, in some embodiments, the plurality of imaging conditions may include one or more of illuminating the sample at different illumination angles, illuminating the sample with different illumination patterns, or illuminating the sample with different wavelengths of light. In some embodiments, the wavelength of light may be selected to match a color of the stain, such as red, pink, or purple.

At block 208 the one or more images of the sample on the slide are processed with a classifier configured to detect the pathogen. The classifier may comprise any suitable classifier such one or more of a machine learning classifier, a neural network, or a convolutional neural network. The classes of organisms may either be pre-defined or taught after generating a digital dataset of particular organism types. For example, a data set of known tagged organism may be used to train the classifier model. The classifier may be trained classify stained specimens such as Gram stained specimens using machine learning-based tools, and the tagged training samples may comprise one or more of blood, plasma, blood and plasma, sterile body fluids, such as cerebrospinal fluid, synovial fluid, pleural fluid and others, sputum and/or mucus, feces, urine, aspirates, biopsies, such as tissue biopsies, swab samples, or other specimens. In some embodiments, the classifier is configured to detect the pathogen with one or more of a color or a morphology of the pathogen. In some embodiments, once the classifier model has been trained, the classifier is configured to detect the pathogen as described herein.

In some embodiments, the classifier may classify any of the types of pathogens described herein. In some embodiments, the classifier may detect clusters of pathogens. In some embodiments, the classifier may detect a subtype of pathogen, such as for bacteria the classifier may classify bacteria into cocci, bacilli, and spiral-shaped, for example. The cocci are round, the bacilli are rods, and the spiral-shaped bacteria can be either rigid (spirilla) or flexible (spirochetes). In some embodiments, the classifier may classify Gram-stained samples as Gram negative or Gram positive. In some embodiments, the classifier may count the number of each type of pathogen. In some embodiments, the image may be annotated or marked to indicate a location and/or type of pathogen detected or suspected. In some embodiments, the classifier may evaluate the morphology of the pathogen to pre-classify the pathogen, such as a bacteria, before using a second classifier to further classify the pathogen into a subtype, such as cocci, bacilli, and spiral-shaped.

In some embodiments, the sample is classified at a rate of at least 15 mm² per minute and optionally one or more of at least 18 mm² per minute, at least 20 mm² per minute, at least 25 mm² per minute, at least 50 mm² per minute, or at least 75 mm² per minute.

The combination of the high resolution and field of view provided by the imaging discussed herein with the analysis and classification provides for analyzing and classifying a high number of cells at the same time. For example, a single image at 100× magnification may include hundreds, thousands, tens of thousands, or even hundreds of thousands of cells that are input to the classifier. The one or more images that are processed with the classifier may comprise a single image or several images that are combined, e.g. with scanning of a conventional microscope across several fields of view, or a plurality of images captured with a computational microscope and combined into a single image with improved resolution as described herein. In some embodiments, the one or more images that are input to the classifier may comprises a scan of an area, which generates several images that are combined and input into the classifier, for example. The analysis and classification may be performed on an entire image. Such analysis and classification can classify hundreds, thousands, tens of thousands, or hundreds of thousands of cells simultaneously in a single image. In some embodiments, at least 1000 cells, at least 10,000 cells, or at least 100,000 cells in a single image are imaged, processed and/or classified at the same time, significantly reducing the time for providing results and/or determining the presence or absence of a pathogen within a sample.

Sensitivity and specificity are measures of a test's, such as a classifier's, ability to correctly classify an image as having a pathogen or not having a pathogen. In some embodiments, sensitivity refers to the classifier's ability to accurately designate an image with pathogen as positive. A highly sensitive test means that there are fewer false negative results, and thus fewer cases where pathogen is missed. In some embodiments, the specificity of a test refers to the classifier's ability to accurately designate an image that does not have a pathogen as negative. A highly specific test means that there are few false positive results. In some embodiments, the classifier is configured to classify the sample at the rates discussed herein while detecting pathogen within the sample with a sensitivity of at least 90%. In some embodiments, the classifier is configured to classify the sample at the rates discussed herein while detecting pathogen within the sample with a sensitivity of at least 95%. The increased area of the one or more samples that are imaged with the resolution generally increases both the sensitivity and specificity, for example when a plurality of samples from a culture medium from a single patient are analyzed.

In some embodiments, the classifier is configured to identify a plurality of cell types with the specificity and sensitivity discussed herein.

In some embodiments, the resolution of the image used in the classifier may be greater than the resolution of a single image obtained in the imaging step. For example, imaging process, such as computational photography, computational imaging, computational microscopy, ptychography or Fourier ptychography may combine multiple images taken under different conditions into a single image with a resolution higher than the constituent images used to generate the combined image. Similarly, in some embodiments, the of images may be acquired with an imaging sensor coupled to a microscope objective having a numerical aperture (NA). In some embodiments, after processing the images, such as through one or more of computational photography, computational imaging, computational microscopy, ptychography or Fourier ptychography and the resolution of the one or more images processed with the classifier corresponds to an effective NA greater than the NA of the microscope objective.

In some embodiments, a sample may be classified while it is still being imaged. For example, in some embodiments, the imaging of the sample at block 206 and the processing of the one or more images at block 208 are performed substantially simultaneously, e.g. at an overlapping time. In some embodiments, imaging of the sample at block 206 and the processing of the one or more images at block 208 are performed simultaneously for at least half of the imaging of the sample and half of the processing of the one or more images of the sample. In some embodiments, the sample is simultaneously imaged at block 206 and classified at block 208 at the rate of at least 15 mm² per minute for at least a portion of the at least 5 mm² of the sample. In some embodiments, at least 30 samples are imaged as described herein with respect to block 206 and processed with the classifier as discussed with respect to block 208 within one hour of receiving the 30 samples, as discussed with respect to block 204.

In some embodiments, scanning and/or imaging at block 206 may be carried out in parallel with analysis and imaging at block 208. For example, a first processor, such as a central processing unit (CPU) or a specialized processor, such as an application specific integrated circuit (ASIC) or graphic processing unit (GPU) or a core or group of cores thereof, may operate the scanning and/or imaging with a second CPU or a specialized processor, such as an ASIC or GPU or a core or group of cores thereof carries out the analysis, such as the classification. In some embodiments, the actions of block 206 and block 208 may be carried out in parallel on different kernels or different process threads. In some embodiments, a first processor, core, or thread, may control the scanning of a first portion of the sample while a second processor, core or thread may carry out analysis of a second portion of the sample. In some embodiments, a first processor, core, or thread, may control analysis for a first pathogen in a sample while a second processor, core or thread may carry out the analysis for a second pathogen in a sample. Carrying out the actions in parallel, as enabled by the subject matter disclosed herein allows for significant reductions in processing times and for quicker access to the results and diagnosis, for example.

In some embodiments, the classification at step 208 is performed with a plurality of separate processes, such as a plurality of threads, that allow the processing with the classifier to be performed more efficiently. For example, the processing with the classifier at step 208 may comprise sub-tasks that are performed in parallel, e.g. on the same or different processors. In some embodiments, the separate classifier processes are allocated to different processor resources such as separate cores of the processor or arranged into sub-tasks that run the processes in parallel on the same processor or different processors. The classifier processes may comprise sub-tasks that can be arranged in a que of sub-tasks to be performed on the same processor or different processor, and combinations thereof.

In some embodiments, a plurality of samples may be processed at overlapping times. For example, a sample may be received, imaged, and processed as described herein with respect to blocks 204, 206, and 208 while a second vile is being cultured and then later processed. For example, a first vial of blood may have been taken from a patient and a blood sample obtained from the first vial and cultured in the culture medium as discussed with respect to block 202. Similarly, a second vial of blood may have been taken from the patient and a second blood sample taken from the second vial and cultured in a second culture medium as disused with respect to block 202. The first blood sample and the second blood sample may be cultured at overlapping times. In some embodiments, the first sample may be received as discussed with respect to block 204, imaged as discussed with respect to block 206, and processed as discussed with respect to block 208 while the second culture medium continues to grow the pathogen. In some embodiments, the first sample may be received as discussed with respect to block 204, imaged as discussed with respect to block 206, and processed as disused with respect to block 208 before the second sample in the second culture medium has been incubated for 40 hours. In some embodiments, the first sample and the second sample may begin incubation within 1 hour, 2 hours, 3 hours, 4 hours, or 12 hours of each other.

In some embodiments, a user may be notified when a classifier detects a pathogen. For example, in some embodiments, an output to a user interface is generated if the classifier detects the pathogen. In some embodiments, an output may be an alert to a user to investigate a finding on the sample detected with the classifier. In some embodiments, the output may be an alert to a user to investigate a finding on the sample detected with the classifier. In some embodiments, the alert may be a visual indicator on the image. In some embodiments, the alert may be an audible alert, for example.

In some embodiments, at block 210, the adequacy of the sample is determined or evaluated, which may be related to the quality of the sample. In some embodiments, an area of the sample is evaluated to determine the adequacy of the sample. For some pathogen and sample types, a sample adequacy can be determined in order to conclude whether the sample and detected pathogen is valid diagnostically. Taking sputum samples as an example, leukocyte and epithelial cells if the count for these cells exceeds certain quantities, the sample may be considered contaminated, hence non-diagnostic. The adequacy can be evaluated with automatic tools based on the digital image produced by the scanner. In some embodiments, since the color of the organisms is considered in Gram analysis, the sample can be evaluated make sure over-staining or under-staining did not occur, which could result in wrong colors of the organisms on the slide, in turn leading to wrong diagnostic conclusions.

The adequacy of the sample can be evaluated and determined in many ways. In some embodiments, the adequacy is evaluated based on one or more of a presence of a type of cell, a density of cells, a thickness of the sample, a staining property of the sample, or a presence of artifacts, for example. In some embodiments, a number of cells, their condition, a ratio between cell populations, the staining of the cells, the morphology of the cells, etc., may be used to determining the quality and/or adequacy of the cells. For example, if a sample has too few or to many cells detected (such as an unrealistically high or low number, above or below an adequacy threshold), that may indicate a low quality or inadequate sample. In some embodiments, a number of cells in an inadequate or low quality condition (such as not intact) may be indicative of an inadequate or low quality sample or image. For example, a ratio of cells in one condition (intact) to another condition (not intact) may indicate a low quality or inadequate sample or image. Counts or ratios of cell populations, different staining conditions (stained, not stained, color of stain), or types of morphology (a first, second, and/or third, or fourth morphology) above or below a threshold may indicate a low quality or inadequate sample.

The adequacy of the sample may be determined or evaluated with respect to one or more of a color metric, a thickness of the sample (greater or less than a desired thickness), a monolayer, or a contamination of the sample. In some embodiment, the adequacy of the sample may be evaluated in response to a color of the sample, such as a color detected in a Gram stained sample. In some embodiments, Gram positive bacteria stain with a violet color, and Gram negative bacteria stain with a red color, and the adequacy of the sample can be determined in response to the colors present in the sample. In some embodiments, the adequacy may be evaluated to determine whether a sample is or stained or under stained, such as stained greater than a desired amount or stained less than a desired amount.

In some embodiments, the adequacy of the sample is determined based on a plurality of cells in the sample detected with the classifier. In some embodiments, the classifier is configured to identify a plurality of cell types and the adequacy is determined based on the plurality of identified cell types. In some embodiments, a plurality of cell types is detected by the classifier, and the adequacy of the sample may be evaluated based on the number of each of the plurality of cell types identified. In some embodiments, the plurality of cell types may include non-pathogenic cell types, for example. The adequacy of such samples may be evaluated based on a number of non-pathogenic cells detected for each of the plurality of non-pathogenic cell types. In some embodiments, the plurality of non-pathogenic cell types may include one or more of hematopoietic cells or epithelial cells.

Although the sample adequacy is described with reference to block 210, the adequacy of the sample may be evaluated at any of the blocks of method 200. In some embodiments, the location of the sample on the slide may be evaluated to determine the adequacy of the sample. In some embodiments, a user such as a remote user is provided with a report to evaluate the adequacy of the sample, for example as part of a decision support system as described herein.

In some embodiments, at block 212 the method 200 may include generating a report of the sample. The values or characteristics in the report may be auto-populated according to the values from the analysis steps. The system may present supporting data from the analysis to aid in the decision or allow additional analysis or amending of the analysis. In some embodiments, such as with multiple slides from the same sample, the system may auto-populate the report values based on rules. For example, if the slides contain different values for the same characteristic, for example one shows abnormal and the other shows normal, the system may choose not to auto-populate the value and alert the user of the discrepancy. In some embodiments, the report may include an indication whether or not one or more pathogens were detected in the sample. For example, in some embodiments, the existence of a pathogen may be reported. In some embodiments, the detection of one or a plurality of pathogens detected in the sample may be reported at block 212. The detection of a pathogen in the sample may be carried out at block 208, for example by analysis of the images with a classifier.

In some embodiments, the method may include using a decision support system for analysis by a user such as a remote user. The decision support system may compare the values of the detected objects with predefined values, which may be default values or determined by the user or center and can be adjusted based on data analysis and suggest if the values are within normal or abnormal range. The user may be given the option to override the suggestion and adjust the values. The decision support system may base its recommendation on properties as described herein such as one or more size, color, number, density, location, morphology, or context, for example. Examples of decision support system recommendations include one or more of: recommend if there is suspected contamination of the sample, recommend severity and type of infection based on organism detections and their formation.

The decision support system may include graphic presentation of certain points of data. For example: presenting the scan with annotations around objects of interest detected, laying out the number/density of organisms by type, formation, context (e.g. inside or outside cells), color or any other characteristic. A decision support system suitable for incorporation in accordance with the present disclosure is described in PCT application no. PCT/IL2021/051329, published as WO2022/097155 on May 12, 2022, entitled “Full field morphology-precise quantification of cellular and sub-cellular morphological events in red/white blood cells” the entire disclosure of which are incorporated herein by this reference.

In some embodiments, an output from processing the one or more images with the classifier is provided in a decision support system. In some embodiments, the sample is received in block 204 at a first location and an output from processing the one or more images with the classifier provided to a second location remote from the first location for analysis by a remote user. In some embodiments, the second location is remote from the first location by being one or more of in a different building, at least 1 kilometer from the first location, or at least 50 kilometers from the first location. In some embodiments, a user interface is configured for a remote user to provide comments and annotations areas of the one or more images.

In some embodiments, the decision support system provides a portion of the one or more images corresponding to a location of a potential pathogen detected with the classifier. In some embodiments, the decision support system compares values of detected cells with reference values and indicates whether the values are within normal range or outside normal range and whether the sample is adequate for analysis. In some embodiments, the decision support system presents a portion of the one or more images with annotations around objects of interest and provides one or more of a number and density of the objects of interest by type, a formation of the objects of interest, a context of the objects of interest, an inside a cell context, an outside a cell context, or a color of the objects of interest.

In some embodiments, a slide is received on a microscope stage from a slide loader at block 204 and an area at least 5 mm² of the slide is imaged at block 206, and the images are processed with the classifier at block 208 automatically to generate an output to a user interface. In some embodiments, the output comprises a first output to a user interface if the classifier detects the pathogen and a second output to the user interface if the classifier does not detect the pathogen.

FIG. 3 is a block diagram of an example computing system 810 capable of implementing one or more of the embodiments described and/or illustrated herein. For example, all or a portion of computing system 810 may perform and/or be a means for performing, either alone or in combination with other elements, one or more of the steps described herein (such as one or more of the steps illustrated in FIG. 2). All or a portion of computing system 810 may also perform and/or be a means for performing any other steps, methods, or processes described and/or illustrated herein. All or a portion of computing system 810 may correspond to or otherwise be integrated with microscope 100 (e.g., one or more of controller 106, memory 108, and/or user interface 112).

Computing system 810 broadly represents any single or multi-processor computing device or system capable of executing computer-readable instructions. Examples of computing system 810 include, without limitation, workstations, laptops, client-side terminals, servers, distributed computing systems, handheld devices, or any other computing system or device. In its most basic configuration, computing system 810 may include at least one processor 814 and a system memory 816.

Processor 814 generally represents any type or form of physical processing unit (e.g., a hardware-implemented central processing unit) capable of processing data or interpreting and executing instructions. In certain embodiments, processor 814 may receive instructions from a software application or module. These instructions may cause processor 814 to perform the functions of one or more of the example embodiments described and/or illustrated herein.

In some embodiments, the classification is performed with a plurality of separate processes, such as a plurality of threads, that allow the processing with the classifier to be performed more efficiently on processor 814, which may comprise a single core processor, a multi core processor, or a plurality of processors, for example. In some embodiments, the separate classifier processes are allocated to different processor resources such as separate cores of the processor 814, or arranged into sub-tasks that run the processes in parallel on the same processor or different processors. The classifier processes may comprise sub-tasks that can be arranged in a que of sub-tasks to be performed on the same processor or different processor, and combinations thereof.

System memory 816 generally represents any type or form of volatile or non-volatile storage device or medium capable of storing data and/or other computer-readable instructions. Examples of system memory 816 include, without limitation, Random Access Memory (RAM), Read Only Memory (ROM), flash memory, or any other suitable memory device. Although not required, in certain embodiments computing system 810 may include both a volatile memory unit (such as, for example, system memory 816) and a non-volatile storage device (such as, for example, primary storage device 832, as described in detail below). In one example, one or more of steps from FIG. 2 may be computer instructions that may be loaded into system memory 816.

In some examples, system memory 816 may store and/or load an operating system 840 for execution by processor 814. In one example, operating system 840 may include and/or represent software that manages computer hardware and software resources and/or provides common services to computer programs and/or applications on computing system 810. Examples of operating system 840 include, without limitation, LINUX, JUNOS, MICROSOFT WINDOWS, WINDOWS MOBILE, MAC OS, APPLE'S IOS, UNIX, GOOGLE CHROME OS, GOOGLE'S ANDROID, SOLARIS, variations of one or more of the same, and/or any other suitable operating system.

In certain embodiments, example computing system 810 may also include one or more components or elements in addition to processor 814 and system memory 816. For example, as illustrated in FIG. 3, computing system 810 may include a memory controller 818, an Input/Output (I/O) controller 820, and a communication interface 822, each of which may be interconnected via a communication infrastructure 812. Communication infrastructure 812 generally represents any type or form of infrastructure capable of facilitating communication between one or more components of a computing device.

Examples of communication infrastructure 812 include, without limitation, a communication bus (such as an Industry Standard Architecture (ISA), Peripheral Component Interconnect (PCI), PCI Express (PCIe), or similar bus) and a network.

Memory controller 818 generally represents any type or form of device capable of handling memory or data or controlling communication between one or more components of computing system 810. For example, in certain embodiments memory controller 818 may control communication between processor 814, system memory 816, and I/O controller 820 via communication infrastructure 812.

I/O controller 820 generally represents any type or form of module capable of coordinating and/or controlling the input and output functions of a computing device. For example, in certain embodiments I/O controller 820 may control or facilitate transfer of data between one or more elements of computing system 810, such as processor 814, system memory 816, communication interface 822, display adapter 826, input interface 830, and storage interface 834.

As illustrated in FIG. 3, computing system 810 may also include at least one display device 824 (which may correspond to user interface 112) coupled to I/O controller 820 via a display adapter 826. Display device 824 generally represents any type or form of device capable of visually displaying information forwarded by display adapter 826. Similarly, display adapter 826 generally represents any type or form of device configured to forward graphics, text, and other data from communication infrastructure 812 (or from a frame buffer, as known in the art) for display on display device 824.

As illustrated in FIG. 3, example computing system 810 may also include at least one input device 828 (which may correspond to user interface 112) coupled to I/O controller 820 via an input interface 830. Input device 828 generally represents any type or form of input device capable of providing input, either computer or human generated, to example computing system 810. Examples of input device 828 include, without limitation, a keyboard, a pointing device, a speech recognition device, variations or combinations of one or more of the same, and/or any other input device.

Additionally or alternatively, example computing system 810 may include additional I/O devices. For example, example computing system 810 may include I/O device 836. In this example, I/O device 836 may include and/or represent a user interface that facilitates human interaction with computing system 810. Examples of I/O device 836 include, without limitation, a computer mouse, a keyboard, a monitor, a printer, a modem, a camera, a scanner, a microphone, a touchscreen device, variations or combinations of one or more of the same, and/or any other I/O device.

Communication interface 822 broadly represents any type or form of communication device or adapter capable of facilitating communication between example computing system 810 and one or more additional devices. For example, in certain embodiments communication interface 822 may facilitate communication between computing system 810 and a private or public network including additional computing systems. Examples of communication interface 822 include, without limitation, a wired network interface (such as a network interface card), a wireless network interface (such as a wireless network interface card), a modem, and any other suitable interface. In at least one embodiment, communication interface 822 may provide a direct connection to a remote server via a direct link to a network, such as the Internet. Communication interface 822 may also indirectly provide such a connection through, for example, a local area network (such as an Ethernet network), a personal area network, a telephone or cable network, a cellular telephone connection, a satellite data connection, or any other suitable connection.

In certain embodiments, communication interface 822 may also represent a host adapter configured to facilitate communication between computing system 810 and one or more additional network or storage devices via an external bus or communications channel. Examples of host adapters include, without limitation, Small Computer System Interface (SCSI) host adapters, Universal Serial Bus (USB) host adapters, Institute of Electrical and Electronics Engineers (IEEE) 1394 host adapters, Advanced Technology Attachment (ATA), Parallel ATA (PATA), Serial ATA (SATA), and External SATA (eSATA) host adapters, Fibre Channel interface adapters, Ethernet adapters, or the like. Communication interface 822 may also allow computing system 810 to engage in distributed or remote computing. For example, communication interface 822 may receive instructions from a remote device or send instructions to a remote device for execution.

In some examples, system memory 816 may store and/or load a network communication program 838 for execution by processor 814. In one example, network communication program 838 may include and/or represent software that enables computing system 810 to establish a network connection 842 with another computing system (not illustrated in FIG. 3) and/or communicate with the other computing system by way of communication interface 822. In this example, network communication program 838 may direct the flow of outgoing traffic that is sent to the other computing system via network connection 842. Additionally or alternatively, network communication program 838 may direct the processing of incoming traffic that is received from the other computing system via network connection 842 in connection with processor 814.

Although not illustrated in this way in FIG. 3, network communication program 838 may alternatively be stored and/or loaded in communication interface 822. For example, network communication program 838 may include and/or represent at least a portion of software and/or firmware that is executed by a processor and/or Application Specific Integrated Circuit (ASIC) incorporated in communication interface 822.

As illustrated in FIG. 3, example computing system 810 may also include a primary storage device 832 and a backup storage device 833 coupled to communication infrastructure 812 via a storage interface 834. Storage devices 832 and 833 generally represent any type or form of storage device or medium capable of storing data and/or other computer-readable instructions. For example, storage devices 832 and 833 may be a magnetic disk drive (e.g., a so-called hard drive), a solid state drive, a floppy disk drive, a magnetic tape drive, an optical disk drive, a flash drive, or the like. Storage interface 834 generally represents any type or form of interface or device for transferring data between storage devices 832 and 833 and other components of computing system 810. In one example, data 835 (which may correspond to the captured images described herein) may be stored and/or loaded in primary storage device 832.

In certain embodiments, storage devices 832 and 833 may be configured to read from and/or write to a removable storage unit configured to store computer software, data, or other computer-readable information. Examples of suitable removable storage units include, without limitation, a floppy disk, a magnetic tape, an optical disk, a flash memory device, or the like. Storage devices 832 and 833 may also include other similar structures or devices for allowing computer software, data, or other computer-readable instructions to be loaded into computing system 810. For example, storage devices 832 and 833 may be configured to read and write software, data, or other computer-readable information. Storage devices 832 and 833 may also be a part of computing system 810 or may be a separate device accessed through other interface systems.

Many other devices or subsystems may be connected to computing system 810. Conversely, all of the components and devices illustrated in FIG. 3 need not be present to practice the embodiments described and/or illustrated herein. The devices and subsystems referenced above may also be interconnected in different ways from that shown in FIG. 3. Computing system 810 may also employ any number of software, firmware, and/or hardware configurations. For example, one or more of the example embodiments disclosed herein may be encoded as a computer program (also referred to as computer software, software applications, computer-readable instructions, or computer control logic) on a computer-readable medium. The term “computer-readable medium,” as used herein, generally refers to any form of device, carrier, or medium capable of storing or carrying computer-readable instructions. Examples of computer-readable media include, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

The computer-readable medium containing the computer program may be loaded into computing system 810. All or a portion of the computer program stored on the computer-readable medium may then be stored in system memory 816 and/or various portions of storage devices 832 and 833. When executed by processor 814, a computer program loaded into computing system 810 may cause processor 814 to perform and/or be a means for performing the functions of one or more of the example embodiments described and/or illustrated herein. Additionally or alternatively, one or more of the example embodiments described and/or illustrated herein may be implemented in firmware and/or hardware. For example, computing system 810 may be configured as an Application Specific Integrated Circuit (ASIC) adapted to implement one or more of the example embodiments disclosed herein.

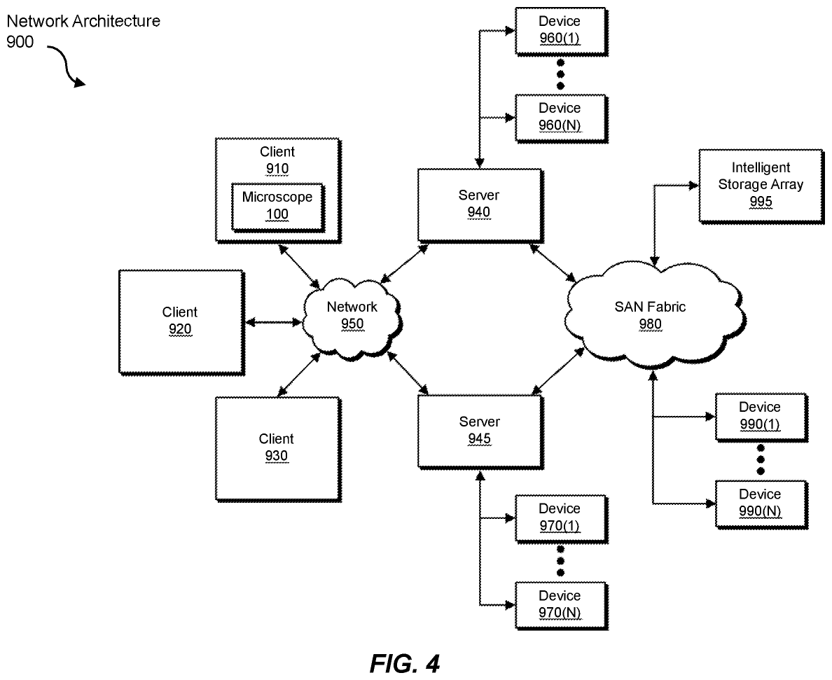

FIG. 4 is a block diagram of an example network architecture 900 in which client systems 910, 920, and 930 and servers 940 and 945 may be coupled to a network 950. As detailed above, all or a portion of network architecture 900 may perform and/or be a means for performing, either alone or in combination with other elements, one or more of the steps disclosed herein (such as one or more of the steps illustrated in FIG. 2). All or a portion of network architecture 900 may also be used to perform and/or be a means for performing other steps and features set forth in the instant disclosure.

Client systems 910, 920, and 930 generally represent any type or form of computing device or system, such as example computing system 810 in FIG. 3. Similarly, servers 940 and 945 generally represent computing devices or systems, such as application servers or database servers, configured to provide various database services and/or run certain software applications. Network 950 generally represents any telecommunication or computer network including, for example, an intranet, a WAN, a LAN, a PAN, or the Internet. In one example, client systems 910, 920, and/or 930 and/or servers 940 and/or 945 may include all or a portion of microscope 100 from FIG. 1.

As illustrated in FIG. 4, one or more storage devices 960(1)-(N) may be directly attached to server 940. Similarly, one or more storage devices 970(1)-(N) may be directly attached to server 945. Storage devices 960(1)-(N) and storage devices 970(1)-(N) generally represent any type or form of storage device or medium capable of storing data and/or other computer-readable instructions. In certain embodiments, storage devices 960(1)-(N) and storage devices 970(1)-(N) may represent Network-Attached Storage (NAS) devices configured to communicate with servers 940 and 945 using various protocols, such as Network File System (NFS), Server Message Block (SMB), or Common Internet File System (CIFS).

Servers 940 and 945 may also be connected to a Storage Area Network (SAN) fabric 980. SAN fabric 980 generally represents any type or form of computer network or architecture capable of facilitating communication between a plurality of storage devices. SAN fabric 980 may facilitate communication between servers 940 and 945 and a plurality of storage devices 990(1)-(N) and/or an intelligent storage array 995. SAN fabric 980 may also facilitate, via network 950 and servers 940 and 945, communication between client systems 910, 920, and 930 and storage devices 990(1)-(N) and/or intelligent storage array 995 in such a manner that devices 990(1)-(N) and array 995 appear as locally attached devices to client systems 910, 920, and 930. As with storage devices 960(1)-(N) and storage devices 970(1)-(N), storage devices 990(1)-(N) and intelligent storage array 995 generally represent any type or form of storage device or medium capable of storing data and/or other computer-readable instructions.

In certain embodiments, and with reference to example computing system 810 of FIG. 3, a communication interface, such as communication interface 822 in FIG. 3, may be used to provide connectivity between each client system 910, 920, and 930 and network 950. Client systems 910, 920, and 930 may be able to access information on server 940 or 945 using, for example, a web browser or other client software. Such software may allow client systems 910, 920, and 930 to access data hosted by server 940, server 945, storage devices 960(1)-(N), storage devices 970(1)-(N), storage devices 990(1)-(N), or intelligent storage array 995. Although FIG. 4 depicts the use of a network (such as the Internet) for exchanging data, the embodiments described and/or illustrated herein are not limited to the Internet or any particular network-based environment.

In at least one embodiment, all or a portion of one or more of the example embodiments disclosed herein may be encoded as a computer program and loaded onto and executed by server 940, server 945, storage devices 960(1)-(N), storage devices 970(1)-(N), storage devices 990(1)-(N), intelligent storage array 995, or any combination thereof. All or a portion of one or more of the example embodiments disclosed herein may also be encoded as a computer program, stored in server 940, run by server 945, and distributed to client systems 910, 920, and 930 over network 950.

As detailed above, computing system 810 and/or one or more components of network architecture 900 may perform and/or be a means for performing, either alone or in combination with other elements, one or more steps of an example method for automated whole-slide scanning of Gram stained slides and early detection of microbiological infection.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor. The processor may comprise a distributed processor system, e.g. running parallel processors, or a remote processor such as a server, and combinations thereof.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

Unless otherwise noted, the terms “connected to” and “coupled to” (and their derivatives), as used in the specification and claims, are to be construed as permitting both direct and indirect (i.e., via other elements or components) connection. In addition, the terms “a” or “an,” as used in the specification and claims, are to be construed as meaning “at least one of.” Finally, for ease of use, the terms “including” and “having” (and their derivatives), as used in the specification and claims, are interchangeable with and shall have the same meaning as the word “comprising.

The processor as disclosed herein can be configured with instructions to perform any one or more steps of any method as disclosed herein.

It will be understood that although the terms “first,” “second,” “third”, etc. may be used herein to describe various layers, elements, components, regions or sections without referring to any particular order or sequence of events. These terms are merely used to distinguish one layer, element, component, region or section from another layer, element, component, region or section. A first layer, element, component, region or section as described herein could be referred to as a second layer, element, component, region or section without departing from the teachings of the present disclosure.

As used herein, the term “or” is used inclusively to refer items in the alternative and in combination.

As used herein, characters such as numerals refer to like elements.

As used herein, the terms “comprise” and “include” are interchangeable.

As used herein, the terms “in response to” and “based on” are interchangeable.

The present disclosure includes the following numbered clauses.

As used herein, the term resolution corresponds to the minimum distance at which an image of lines on a resolution target can be separated.

The present disclosure includes the following numbered clauses.

Clause 1. A method of processing a sample to detect a pathogen, the method comprising: receiving a slide with the sample on the slide, wherein the sample has been stained with one or more of a Gram stain, an Acid-Fast stain, or a Giemsa stain; imaging at least 5 mm² of the sample at a rate of at least 15 mm² per minute at a resolution of 0.3 μm or better to generate one or more images; and processing the one or more images of the sample with a classifier configured to detect the pathogen in the sample.

Clause 2. The method of clause 1, wherein the imaging of the sample and the processing of the one or more images are performed simultaneously and optionally performed simultaneously for at least half of the imaging of the sample and half of the processing of the one or more images of the sample.

Clause 3. The method of clause 2, wherein the sample is imaged and classified at the rate of at least 15 mm² per minute for at least a portion of the at least 5 mm² of the sample.

Clause 4. The method of clause 2, wherein a first processor, core, or thread, generates a first portion of the one or more images from a first portion of the sample while a second processor, core or thread processes, with the classifier, a second portion of the one or more images from a second portion of the sample.

Clause 5. The method of clause 2, wherein a first processor, core, or thread, performs analysis for a first pathogen in the sample while a second processor, core or thread performs analysis for a second pathogen in the sample.

Clause 6. The method of clause 1, wherein the sample is classified at a rate of at least 15 mm² per minute and optionally one or more of 18 mm² per minute, 20 mm² per minute, 25 mm² per minute, 50 mm² per minute, or 75 mm² per minute.

Clause 7. The method of clause 1, wherein the rate comprises at least 18 mm² per minute and optionally one or more of at least 20 mm² per minute, at least 25 mm² per minute, 50 mm² per minute, or 75 mm² per minute.

Clause 8. The method of clause 1, wherein the at least 5 mm² of the sample that is imaged comprises one or more of at least 7.5 mm², at least 10 mm², at least 20 mm², at least 30 mm², at least 50 mm², or at least 70 mm².

Clause 9. The method of clause 1, wherein of the one or more images of the sample have one or more of at least 1,000 cells, at least 10,000 cells, or at least 100,000 cells that are processed with the classifier.

Clause 10. The method of clause 1, wherein cells of the one or more images of the sample are processed with the classifier at a rate of at least 1.000 cells per minute, at least 10,000 cells per minute, or at least 100,000 cells per minute.

Clause 11. The method of clause 1, wherein the resolution comprises 0.25 μm or better and optionally 0.22 μm or better.

Clause 12. The method of clause 1, wherein a plurality of samples on a plurality of slides are received to generate one or more images for each of the plurality of samples.

Clause 13. The method of clause 12, wherein the plurality of samples has been taken from different patients.

Clause 14. The method of clause 12, wherein a subset of the plurality of samples has been taken from a single patient and optionally wherein the subset comprises at least two samples.

Clause 15. The method of clause 12, wherein the plurality of samples comprises at least 30 samples and wherein at least 30 samples are imaged and processed with the classifier within one hour.

Clause 16. The method of clause 1, wherein an output to a user interface is generated if the classifier detects the pathogen.

Clause 17. The method of clause 16, wherein the output comprises an alert to a user to investigate a finding on the sample detected with the classifier.

Clause 18. The method of clause 1, wherein the classifier comprises one or more of a machine learning classifier, a neural network, or a convolutional neural network.

Clause 19. The method of clause 18, wherein the classifier is configured to detect the pathogen with one or more of a color or a morphology of the pathogen.

Clause 20. The method of clause 1, wherein the sample has been stained with the Gram stain.

Clause 21. The method of clause 1, wherein the sample has been stained with the Acid-Fast stain.

Clause 22. The method of clause 1, wherein the sample has been stained with the Giemsa stain.

Clause 23. The method of clause 1, wherein the classifier is configured to detect the pathogen with a sensitivity of at least 90% and optionally at least 95%.

Clause 24. The method of clause 1, wherein the classifier is configured to detect the pathogen with a specificity of at least 90% and optionally at least 95%.

Clause 25. The method of clause 1, wherein the one or more images are generated with one or more of computational photography, computational imaging, computational microscopy, ptychography or Fourier ptychography.

Clause 26. The method of clause 25, wherein the one or more images are generated from a plurality of images captured with a plurality of imaging conditions.

Clause 27. The method of clause 26, wherein the different illumination condition comprises one or more of a different illumination angle, a different illumination pattern or a different wavelength.

Clause 28. The method of clause 26, wherein the resolution of the one or more images processed with the classifier is finer than a resolution of each of the plurality of images.

Clause 29. The method of clause 26, wherein the plurality of images is acquired with a microscope objective having a numerical aperture (NA) and wherein the resolution of the one or more images processed with the classifier corresponds to an effective NA greater than the NA of the microscope objective.

Clause 30. The method of clause 1, wherein the pathogen comprises one or more of a bacterium, a fungus or a yeast and the sample comprises a cultured blood sample.

Clause 31. The method of clause 1, wherein the sample comprises a cultured sample, in which a blood sample has been cultured in a culture medium to grow the pathogen.

Clause 32. The method of clause 31, wherein the blood sample has been cultured in a culture medium for less than 48 hours to grow the pathogen in the cultured sample and optionally no more than 24 hours.

Clause 33. The method of clause 32, wherein the blood sample has been cultured in the culture medium for no more than one or more of 1 hour, 2 hours, 4 hours, 8 hours, or 16 hours.

Clause 34. The method of clause 32, wherein a first vial of blood has been taken from a patient and the blood sample has been obtained from the first vial and cultured in the culture medium and wherein a second vial of blood has been taken from the patient, a second blood sample taken from the second vial and cultured in a second culture medium and wherein the first blood sample and the second blood sample are cultured at overlapping times and wherein the receiving, the imaging and the processing of the sample from the first vial are performed while the second culture medium continues to grow the pathogen.

Clause 35. The method of clause 34, wherein the sample is received, imaged and processed before the second sample in the second culture medium has been incubated for 40 hours.

Clause 36. The method of clause 35, wherein the blood sample from the first vial has been cultured in the culture medium for no more than one or more of 1 hour, 2 hours, 4 hours, 8 hours, or 16 hours.

Clause 37. The method of clause 1, wherein an adequacy of the sample is evaluated.

Clause 38. The method of clause 37, wherein the adequacy of the sample is evaluated with one or more of a color metric, a thickness of the sample, a monolayer, or a contamination of the sample.

Clause 39. The method of clause 37, wherein the sample adequacy is evaluated in response to a color of the sample and optionally wherein the stain comprises a Gram stain.

Clause 40. The method of clause 39, wherein the sample adequacy is evaluated to determine overstaining or under staining of the sample.

Clause 41. The method of clause 37, wherein the adequacy is evaluated based on one or more of a presence of a type of cell, a density of cells, a thickness of the sample, a staining property of the sample, or a presence of artifacts.

Clause 42. The method of clause 41, wherein the classifier is configured to identify a plurality cell types and the adequacy is evaluated based on the plurality of identified cell types.

Clause 43. The method of clause 42, wherein a number of each of the plurality of cell identified cell types is determined and the adequacy of the sample is determined based on the number of said each of the plurality of identified cell types.

Clause 44. The method of clause 42, wherein the plurality of cell types comprises a plurality of non-pathogenic cell types and optionally wherein the plurality of non-pathogenic cell types comprises one or more of hematopoietic cells or epithelial cells.

Clause 45. The method of clause 33, wherein an area of the sample on the slide is evaluated to determine the adequacy of the sample.

Clause 46. The method of any one of clauses 37 to 45, wherein the adequacy is determined based on a number of cells having a condition, morphology, or stain.

Clause 47. The method of clause 46, wherein the condition is intact or not intact, the morphology is morphology of a first type of a plurality to types, or the stain is a first type of a plurality of types of stains.

Clause 48. The method of any one of clauses 37 to 45, wherein the adequacy is determined based on a ratio of cells having a condition, morphology, or stain.

Clause 49. The method of clause 48, wherein the ratio is a ratio of cells in a first condition to a ratio of cells in a second condition, the ratio is a ratio of cells having a first morphology to cells having a second morphology, or the ratio is a ratio of cells having first type of stain to cells having a second type of stain.

Clause 50. The method of clause 1, wherein a report of the sample is generated.

Clause 51. The method of clause 50, wherein the report includes auto-populated values and presents supporting data to a user to allow the data to review the supporting data and amend the report.

Clause 52. The method of clause 1, wherein the sample is received at a first location and an output from processing the one or more images with the classifier provided to a second location remote from the first location for analysis by a remote user and optionally wherein the second location is remote from the first location by being one or more if in a different building or at least 1 kilometer from the first location and optionally at least 50 kilometers.

Clause 53. The method of clause 52, wherein a user interface is configured for a remote user to provide comments and annotations areas of the one or more images.

Clause 54. The method of clause 1, wherein an output from processing the one or more images with the classifier is provided in a decision support system.

Clause 55. The method of clause 54, wherein the decision support system provides a portion of the one or more images corresponding to a location of a potential pathogen detected with the classifier.

Clause 56. The method of clause 54, wherein the decision support system compares values of detected cells with reference values and indicates whether the values are within normal range or outside normal range and whether the sample is adequate for analysis.

Clause 57. The method of clause 58, wherein the decision support system presents a portion of the one or more images with annotations around objects of interest, and provides one or more of a number and density of the objects of interest by type, a formation of the objects of interest, a context of the objects of interest, an inside a cell context, an outside a cell context, or a color of the objects of interest.

Clause 58. The method of clause 1, wherein the slide is received on a microscope stage from a slide loader and wherein receiving the slide, imaging the at least 5 mm², and processing the one or more images with the classifier are performed automatically to generate an output to a user interface.

Clause 59. The method of clause 58, wherein the output comprises a first output to a user interface if the classifier detects the pathogen and a second output to the user interface if the classifier does not detect the pathogen.

Clause 60. The system of clause 1, wherein the sample has been obtained from one or more of a blood, a plasma sample, a bodily fluid, a cerebrospinal fluid, a synovial fluid, a pleural fluid, a sputum, a mucus, an excrement, urine, an aspirate, a biopsy, or a swab and optionally wherein the sample comprises a cultured sample.

Clause 61. The method of clause 1, wherein the sample comprises a cultured sample exposed to an antibiotic to perform an antibiotic sensitivity test.

Clause 62. The method of clause 1, wherein the classifier classifies the pathogen in the one or more images.

Clause 63. An apparatus comprising: a processor configured to perform the method of any one of the preceding clauses.

Embodiments of the present disclosure have been shown and described as set forth herein and are provided by way of example only. One of ordinary skill in the art will recognize numerous adaptations, changes, variations and substitutions without departing from the scope of the present disclosure. Several alternatives and combinations of the embodiments disclosed herein may be utilized without departing from the scope of the present disclosure and the inventions disclosed herein. Therefore, the scope of the presently disclosed inventions shall be defined solely by the scope of the appended claims and the equivalents thereof.

1. A method of processing a sample to detect a pathogen, the method comprising:
receiving a slide with the sample on the slide, wherein the sample has been stained with one or more of a Gram stain, an Acid-Fast stain, or a Giemsa stain;
imaging at least 5 mm² of the sample at a rate of at least 15 mm² per minute at a resolution of 0.3 μm or better to generate one or more images; and
processing the one or more images of the sample with a classifier configured to detect the pathogen in the sample.

2. The method of claim 1, wherein the imaging of the sample and the processing of the one or more images are performed simultaneously and optionally performed simultaneously for at least half of the imaging of the sample and half of the processing of the one or more images of the sample.

3. The method of claim 2, wherein the sample is imaged and classified at the rate of at least 15 mm² per minute for at least a portion of the at least 5 mm² of the sample.

4. The method of claim 2, wherein a first processor, core, or thread, generates a first portion of the one or more images from a first portion of the sample while a second processor, core or thread processes, with the classifier, a second portion of the one or more images from a second portion of the sample.

5. The method of claim 2, wherein a first processor, core, or thread, performs analysis for a first pathogen in the sample while a second processor, core or thread performs analysis for a second pathogen in the sample.

6. The method of claim 1, wherein the sample is classified at a rate of at least 15 mm² per minute and optionally one or more of 18 mm² per minute, 20 mm² per minute, 25 mm² per minute, 50 mm² per minute, or 75 mm² per minute.

7. The method of claim 1, wherein the rate comprises at least 18 mm² per minute and optionally one or more of at least 20 mm² per minute, at least 25 mm² per minute, 50 mm² per minute, or 75 mm² per minute.

8. The method of claim 1, wherein the at least 5 mm² of the sample that is imaged comprises one or more of at least 7.5 mm², at least 10 mm², at least 20 mm², at least 30 mm², at least 50 mm², or at least 70 mm².

9. The method of claim 1, wherein the one or more images of the sample have one or more of at least 1,000 cells, at least 10,000 cells, or at least 100,000 cells that are processed with the classifier.

10. The method of claim 1, wherein cells of the one or more images of the sample are processed with the classifier at a rate of at least 1,000 cells per minute, at least 10,000 cells per minute, or at least 100,000 cells per minute.

11. The method of claim 1, wherein the resolution comprises 0.25 μm or better and optionally 0.22 μm or better.

12. The method of claim 1, wherein a plurality of samples on a plurality of slides are received to generate one or more images for each of the plurality of samples.

13. The method of claim 12, wherein the plurality of samples has been taken from different patients.

14. The method of claim 12, wherein a subset of the plurality of samples has been taken from a single patient and optionally wherein the subset comprises at least two samples.

15. The method of claim 12, wherein the plurality of samples comprises at least 30 samples and wherein at least 30 samples are imaged and processed with the classifier within one hour.

16. The method of claim 1, wherein an output to a user interface is generated if the classifier detects the pathogen.

17. The method of claim 16, wherein the output comprises an alert to a user to investigate a finding on the sample detected with the classifier.

18. The method of claim 1, wherein the classifier comprises one or more of a machine learning classifier, a neural network, or a convolutional neural network.

19. The method of claim 18, wherein the classifier is configured to detect the pathogen with one or more of a color or a morphology of the pathogen.

20. The method of claim 1, wherein the sample has been stained with the Gram stain.

21.-63. (canceled)
